(19) United States
(12) Patent Application Publication
(10) Pub. No.: US 2008/0249020 A1
(43) Pub. Date: Oct. 9, 2008

(54) Human Growth Hormone Treatment Methods

(75) Inventors: George Bright, San Mateo, CA (US); Bob (Robert) An, Bridgewater, NJ (US)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/118,256

(22) Filed: May 9, 2008

Related U.S. Application Data
(63) Continuation of application No. 11/127,705, filed on May 12, 2005.
Application No. 11/127,705 is a continuation-in-part of application No. PCT/DK2003/000780, filed on Nov. 13, 2003.
(60) Provisional application No. 60/426,024, filed on Nov. 13, 2002.

(51) Int. Cl. A61K 38/27; A61P 43/00

(52) U.S. Cl. 514/12

(57) ABSTRACT

New methods for developing dosing formulas that allow the determination of appropriate dosage regimens for human growth hormone (hGH) compounds; methods for using such formulas to calculate individualized hGH compound dosage regimens; methods for treatment of hGH-responsive syndromes in patients using such determined regimens; systems, devices, and computer-readable media comprising instructions for receiving predictive value information and for generating dosage regimens and/or dosing formulas; and other related systems and methods are provided by the invention described herein.

### CROSS-REFERENCE TO RELATED APPLICATIONS

This patent application is a continuation of U.S. application Ser. No. 11/127,705 filed May 12, 2005 which is a continuation-in-part of International (PCT) Patent Application PCT/DK03/00780, published in English as WO 2004/043484, designating the United States of America (“US”), which was filed Nov. 13, 2003, claiming priority under 35 U.S. C. § 119 from U.S. Provisional Patent Application 60/426,024, filed Nov. 13, 2002 (to which priority is also claimed from this application), the content of both of which is hereby expressly incorporated by reference.

### FIELD OF THE INVENTION

The present invention relates to methods for achieving an optimum clinical response to human growth hormone therapy.

### BACKGROUND OF THE INVENTION

About 100,000 children worldwide are treated with recombinant human growth hormone (rhGH) to achieving optimum growth, but dosing remains largely arbitrary. The lack of evidence-based practices regarding the dosing of rhGH in pediatric GH deficiency limits the treatment options available.

Thus, there is a need in the art for rationalized, individualized dosing regimens for rhGH and similar growth hormone (GH) compositions.

### SUMMARY OF THE INVENTION

In one aspect, the present invention provides methods for treating a GH-responsive patient. The methods are carried out by, among other things, performing the steps of:

(i) measuring a value for one or more predictive variables, including, without limitation, height, target height, growth velocity, gender, bone age, plasma insulin-like-growth factor 1 (IFG-1) levels, parental height, pubertal status, genotype, health status, and environmental factors, to obtain a predictive profile for a patient,

(ii) applying to the predictive profile a hGH dosing formula in order to determine a dosing regimen; and

(iii) administering hGH to the patient according to the determined regimen.

In another aspect, the invention provides methods for determining a hGH dosing regimen for a patient, which are carried out by, among other things, performing the steps of:

(i) measuring the values for one or more predictive variables in the patient; and

(ii) applying the measured values to a hGH-dosing formula to determine a hGH dosing regimen for the patient.

In another aspect, the present invention provides methods for developing dosing formulas for hGH, which are carried out by the steps of:

(i) measuring the values of one or more candidate predictive variables in a plurality of patients;

(ii) administering different dosages of hGH to subsets of said patients;

(iii) monitoring one or more parameters of desired outcome in said patients over time; and

(iv) performing multiple regression analysis to identify predictive variables and/or predictive sets of variables, thereby producing a formula (which may be referred to as a “hGH dosing formula”) that expresses the relationship between the variables and/or sets of variables and an optimum hGH dosage regimen. In some embodiments, the invention further comprises:

(v) repeating steps (ii)-(iv) in order to validate and/or refine a previously determined hGH dosing formula. The invention also provides methods that comprise altering an existing hGH dosing formula by repeating the analysis using updated data sets, as well as developing different formulas tailored to specific sub-populations of patients.

In another aspect, the invention encompasses a system for determining optimal hGH dosing, which comprises a processor programmed to apply the values for predictive variables as measured in a patient a hGH-dosing formula, resulting in the calculation of an appropriate hGH dosing regimen.

### DETAILED DESCRIPTION OF THE INVENTION

The present invention is based on, among other things, the finding that optimum dosing of human growth hormone (hGH) in hGH-responsive patients can be achieved by quantitative evaluation of a plurality of clinical variables. The invention provides methods for developing dosing formulas that allow the determination of appropriate dosage regimens; methods for using the formulas to calculate individualized dosage regimens; and methods for treatment of hGH-responsive syndromes in a patient using the determined regimens.

As used herein, hGH refers to wild-type human growth hormone (i.e., a polypeptide having the amino acid sequence of hGH 1-191 as disclosed, e.g., in Hormone Drugs, Gueriguian et al., USP Convention, Rockville Md. 1982), whether naturally produced or recombinantly produced (i.e., rhGH), as well as analogues and derivatives of hGH and hGH analogues that are clinically useful because they exhibit growth-promoting biological activity. Analogues include, without limitation, variant hGH polypeptides having an amino acid sequence that differs from the sequence of wild-type hGH by insertion, deletion, and/or substitution of one or more amino acids. Derivatives include, without limitation, wild-type or variant hGH polypeptides that have undergone one or more chemical modifications, including, e.g., conjugation of peptide or non-peptide moieties to the “backbone” wild-type or variant hGH amino acid sequence. Preferably, variants and analogues exhibit at least about 25% of the bioactivity of wild-type hGH, more preferably at least about 50%, and most preferably at least about 75% of the bioactivity of wild-type hGH. Wild-type hGH bioactivity has been well characterized in scientific literature and patent documents.

As used herein, a hGH-responsive syndrome refers to any clinical situation that can benefit from hGH administration, including, without limitation, hGH deficiency, delayed or impaired growth, inflammatory diseases, autoimmune diseases, and viral syndromes such as, e.g., AIDS.

As used herein, a hGH-dosing regimen refers to any aspect of the therapeutic administration of hGH, including, without limitation, the amount of hGH administered with each administration; dosing schedule; the mode of administration; and combination therapies. Agents that may be combined with hGH include, without limitation, androgens, estrogens, glucocorticoids, thyroid hormone, IGF-1, and one or more IGF-1 binding proteins. In some embodiments, the dosage regimen comprises hGH dosage amount and dosage schedule. In some embodiments, the dosage regimen comprises hGH dosage amount, dosage schedule, and mode of administration. In some embodiments, the dosage regimen comprises dosage amount and dosage schedule of hGH in combination with another pharmaceutically active agent. An appropriate dosing regimen is one that is predicted to result in the desired clinical outcome, such as, e.g., growth rate, degree of bone mineralization, etc.

A predictive variable as used herein refers to an identifiable feature of the patient that, either alone or in combination with other variables, is used in a dosing formula to calculate an appropriate hGH dosage regimen. Non-limiting examples of predictive variables include height; target height; growth velocity; gender; bone age; parental height; plasma levels of insulin-like-growth factor 1 (IGF-1), hGH, and/or other hormones, growth factors, binding proteins, and the like; pubertal status; overall health status; environmental factors; and genotype. Predictive variables may also include any identifiable aspect of the functioning of any signal transduction and gene transcription pathways, including, e.g., those that mediate responses to hGH or to other relevant hormones or growth factors.

One or more predictive variables may form a “predictive profile” for an individual. For example, a predictive variable may form part of a set of variables that, in particular combinations of values, are, in the aggregate, predictive of appropriate hGH dosing regimens. Alternatively, a single predictive variable may also be predictive of hGH dosing regimens in certain individuals.

The following is a non-limiting list of exemplary predictive values and a description of how such values are expected to be commonly used in, inter alia, practicing the dosage regimen-profiling and profile-generating aspects of the invention.

Height, expressed in centimeters, refers to the height of the patient at presentation. Preferably, height is determined using a Harpenden stadiometer or similar device.

Target height, expressed in centimeters, refers to the desired height.

Growth velocity is expressed as cm/y.

Bone age, expressed in years and months, is determined using radiographic analysis interpreted using one or more sets of standards (Pyle et al., Am J. Phys. Anthropol. 35:331, 1971).

IGF-1 levels, expressed as ng/ml or as the natural logarithm (ln) of this value, may be determined by any method known in the art, such as, e.g., by radioimmunoassay (Rosenfeld et al., J. Pediatr 109:428, 1986).

Pubertal status is determined by clinical observation and/or plasma sex steroid determination.

Health status of the patient include, without limitation, the presence or absence of identifiable diseases or syndromes, including those unrelated to the patient's underlying hGH profile.

Environmental factors include, without limitation, nutrition, and any factor related to, or caused by, where the patient lives, including, e.g., exposure to pollutants, overall climate, amount of sunlight, and the like.

Genotype refers to any feature of a patient's genetic complement that is identifiable by conventional means, such as those reflected, e.g., in changes in chromosome structure and/or nucleotide sequence. In one embodiment, the genotype feature for use in methods of the present invention is presence or absence of one or more genetic polymorphisms. Genetic polymorphism refers to a variation among individuals in the nucleotide and/or amino acid sequence of a gene or protein, respectively. Such polymorphisms may be identified using methods known in the art, including, e.g., sequencing, hybridization, and restriction analysis techniques. Relevant polymorphisms include, without limitation, polymorphisms in genes and proteins involved in growth, such as, e.g., hormones, growth factors, hormone and growth factor receptors, signaling pathways, and the like, such as, for example, the Arg231-Cys substitution in Gsa.

### Use of Predictive Profiles for Determination of Optimum hGH Dosing Regimen:

As used herein, a hGH-dosing formula is a means of calculating an appropriate dosing regimen using patient-specific clinical features that form a patient's predictive profile.

In practicing the present invention, dosing formulas can be developed and refined by iterative analysis of the predictive value of patient-specific clinical features in determining optimal growth outcomes following hGH treatment. Randomized patient populations are subjected to an initial measurement of candidate predictive variables, after which different groups receive different doses of hGH and are monitored periodically. Outcome measurements may be one or more of: change or retention of height; bone mineral density, bone volume, lean body mass; fat mass, or any other suitable clinical measurement, following various times of treatment. Modalities used to perform these measurements include, without limitation, computerized tomography (CT) and dual emission X-ray absorptiometry (DEXA).

Multivariate regression analysis of the results obtained by relevant analysis can be performed using conventional methods, such as, e.g., the SAS program (SAS Institute, Inc., Gary N.C.). Stepwise analysis can be performed to identify predictive variables, and the interactions between independent variables are evaluated. The analysis may involve the operator specifying the form of the values (e.g., linear, logarithmic, or exponential); the use of convergence rules and model types; and p limits. After the relationship between hGH dose and the selected variables is established, the formula may be validated by assessing the distribution and magnitude of residuals (difference between the observed dose and the dose predicted from the model) using separate data sets.

In one embodiment, the predictive variables are target height change, bone age, IGF-1 level, and paternal height. In another embodiment, the variables are gender, target height change, bone age, IGF-1 level, and paternal height. In yet another embodiment, the variables are gender, target height change, IGF-1 level, bone age, paternal height, and pubertal status. Type of hGH compound also may be a variable. It will be understood that any combination of these and/or other variables may be used following validation.

A non-limiting example of a useful dosing algorithm is: Dose=(0.022×desired height change)+(0.00426×bone age)+(0.0085×ln IGF-1)−(0.00071×paternal height).

The present invention encompasses the formulation of different dosing algorithms that are tailored to different sub-populations of patients, such as, e.g., male and female patients.

### Methods of Treatment:

A dosing algorithm obtained by inventive methods provided herein can be advantageously used in clinical settings for planning the therapeutic administration of hGH. Typically, a patient is examined and samples are obtained for analyses of patient-specific variables. The data obtained from these analyses are entered into a device comprising a computer processor programmed to apply (comprising instructions for application of) a dosing algorithm and thereby calculate an individualized dosing regimen appropriate for that patient (and provide such regimen to a user of the device). The invention also encompasses iterative dosing; i.e., monitoring changes in predictive variables in a particular patient once treatment has been initiated, followed by re-calculation of a dosing regimen.

Pharmaceutical compositions or formulations of hGH for use in the present invention typically comprise hGH in combination with one or more pharmaceutically acceptable excipients or adjuvants. Administration may be by any suitable means, including, without limitation, via subcutaneous, intravenous, transmucosal, or oral routes, or by inhalation. The most preferable mode of administration to use for a particular patient is one aspect of an appropriate dosing regimen that may be identified using the methods of the invention.

### System for Calculating Dosage Regimens:

The present invention provides encompasses a system for determining optimal hGH dosing, which comprises a processor programmed to apply to the values for predictive variables as measured in a patient a hGH-dosing formula, resulting in the calculation of an appropriate hGH dosing regimen. The processor may be any type of computational device, and preferably is a Pocket Personal Computer (“PPC”). Thus, in another aspect, the invention provides computer-readable media comprising computer-readable instructions for obtaining predictive values for determining a profile and/or for applying entered predictive values to obtain an appropriate hGH dosing regiment.

The PPC may be any intelligent portable device, including—but not limited to—a personal digital assistant, a programmable cellular phone, a smart medical device (such as a drug delivery device or a medical monitor), or any other portable device that has a processor and memory, an input means, and a display means. Preferably, the PPC is a handheld electronic device with bi-directional communication capability. It may have wireless communication capabilities or it may have a interface for connecting it to a standard wired network, existing phone system, LAN, WAN, or the like.

The processor is preferably capable of being programmed to prompt the user for certain information and to input certain data related to that information via the input means, process the data, and store the data locally. In one embodiment, the PPC may be a standard digital personal assistant running on the Window CE software, such as, e.g., HP Jornada 560 personal digital assistant (Product no: F2915A version ABA A1Q3 using Window CE ver. 1.0-1.1).

All patents, patent applications, and literature references referred to herein are hereby incorporated by reference in their entirety.

Many variations of the present invention will suggest themselves to those skilled in the art in light of the above detailed description. Such obvious variations are within the full intended scope of the appended claims.

1. A method for treating a human growth hormone (hGH)-responsive syndrome in a patient in need of such treatment comprising:
(i) measuring the values for one or more predictive variables in the patient to obtain a predictive profile for the patient;
(ii) applying to the predictive profile a hGH-dosing formula to determine a hGH dosing regimen; and
(iii) administering hGH to the patient according to the determined dosing regimen.

2. The method of claim 1, wherein the predictive variables are selected from the group consisting of: height; target height; growth velocity; gender; bone age; parental height; plasma levels of insulin-like-growth factor 1 (IGF-1; pubertal status; overall health status; environmental factors; and genotype.

3. The method of claim 1, wherein the predictive variables are desired height change, bone age, IGF-1 level, and paternal height.

4. The method claim 3, wherein the hGH-dosing formula is the algorithm dose=(0.022×desired height change)+(0.00426×bone age)+(0.0085×ln IGF-1)−(0.00071×paternal height).

5. A method for determining a hGH dosing regimen for a patient, the method comprising:
(i) measuring the values for one or more predictive variables in the patient; and
(ii) applying the measured values to a hGH-dosing formula to determine a hGH dosing regimen for the patient.

6. The method of claim 5, wherein the predictive variables are selected from the group consisting of: height, target height, gender, bone age, plasma insulin-like-growth factor 1 (IFG-1) levels, parental height, and pubertal status.

7. A system for determining hGH dosing regimen comprising a processor programmed to apply a hGH-dosing formula to one or more predictive values measured in a patient.
